(19) United States
(12) Patent Application Publication
(10) Pub. No.: US 2021/0325299 A1
(43) Pub. Date: Oct. 21, 2021

(54) DIGITAL ASSESSMENT OF CHEMICAL DIP TESTS

(71) Applicant: ADEY HOLDINGS (2008) LIMITED, Cheltenham (GB)

(72) Inventors: Cameron Hardie, Cheltenham (GB); Helen Isherwood, Cheltenham (GB); Mo Jassal, Cheltenham (GB); Shailesh Prakash, Cheltenham (GB); Neil Watson, Cheltenham (GB); James White, Cheltenham (GB)

(21) Appl. No.: 17/274,080

(22) PCT Filed: Aug. 29, 2019
(86) PCT No.: PCT/EP2019/073065

§ 371 Date: Mar. 5, 2021

(30) Foreign Application Priority Data
Sep. 7, 2018 (GB) 1814621.7

(51) Int. Cl. G01N 21/27

(52) U.S. Cl. CPC G01N 21/27; G01N 21/80

(57) ABSTRACT

A method of digital assessment of chemical dip tests is disclosed. A photograph of a dip tester including a colour change pad is taken with a mobile device, for example a mobile phone. The dip tester is photographed on or alongside a reference card, with both the dip tester and the reference card in the same frame. A processor on the mobile device analyses the photograph thus obtained to determine a value for a characteristic of water being tested. The characteristic may be for example, pH, concentration of iron, concentration of copper. The water may be central heating system water.

The present invention relates to digital assessment of chemical dip tests, particularly to assessment of dip tests conducted on central heating and/or cooling system water, using a mobile device including a digital camera.

### BACKGROUND TO THE INVENTION

Chemical dip tests are known, and various types are available for, among other things, testing pH and the presence and concentration of a variety of chemicals. In particular it is known to test central heating and/or cooling system water for pH and concentration of iron, copper, and a corrosion inhibitor (for example a molybdate). “Dip tests” are Zo available for testing all of these things. A dip test is typically in the form of a pad impregnated with a reagent, which in turn is mounted on a stick to act as a carrier. The pad is dipped in a sample of the liquid to be tested, and the impregnated pad then changes colour. The colour of the pad can be compared to a reference to determine the concentration of the particular chemical being tested for, the pH value, etc.

One of the problems with this type of test is the inconsistency introduced by the human comparison between the dipped pad and the colour reference. Even experienced technicians can make mistakes, since the difference in colour between one result and a materially different result can be subtle.

It is also known to use digital cameras, particularly cameras on mobile smartphones, and appropriate software in order to capture a colour sample, and by comparing it to a reference in the same captured frame, attempt to objectively identify the colour of the sample. This sort of technology has been used for example to mix paint to match an existing colour, or to identify skin colour and identify matching make-up.

However this technology is also prone to inaccuracy and in adverse lighting conditions can often misidentify a colour by a considerable degree.

### SUMMARY OF THE INVENTION

According to a first aspect of the present invention, there is provided a method of testing central heating and/or cooling water comprising the steps of:
providing at least one colour-change dip tester for measuring a characteristic to be tested, and dipping the colour-change dip tester in the central heating and/or cooling water;
providing a colour reference card, the colour reference card having a range of reference colours on its surface, the reference colours on the colour reference card corresponding to a range of possible colours of a dipped dip tester;
using a digital camera, taking a photograph of the dipped dip tester and the colour reference card;
by a processor, identifying an area of the digital photograph corresponding to an image of the colour-change portion of the dip tester, and determining a colour of that area of the photograph;
by the processor, identifying areas in the digital photograph corresponding to an image of the reference colours on the colour reference card, and determining colours associated with reference areas;
determining the closest of the colours associated with reference areas to the colour of the image of the colour-change portion of the dip tester, and determining on that basis a value of the characteristic to be tested.

The characteristic to be tested may be for example, pH, concentration of iron, concentration of copper, concentration of aluminium, and/or concentration of a corrosion inhibitor (for example a molybdate). In some embodiments, multiple colour-change dip testers may be provided for testing multiple characteristics. The multiple dip testers may be mounted on a single carrier, for easily carrying out multiple tests in one operation. Likewise, the colour reference card may include multiple colour ranges corresponding with multiple types of dip test.

In some cases, multiple dip tests may be provided for the same characteristic, for example it is already known to provide dual- or triple-pad dip tests for testing pH, where each pad is designed to provide a clear colour difference for a particular range of values of pH.

Preferably, the colour reference card includes indicia indicating how to position the dipped tester in relation to the reference colours. Ideally, the dipped tester is placed on the colour reference card, in an indicated location, so that the position of the colour-change portion of the dip tester in relation to the reference colours is predetermined.

Preferably, the colour reference card includes registration marks to aid determination by the processor of the parts of the image relating to the colour-change pad, and the parts of the image relating to the reference colours. In one embodiment, there are four registration marks substantially at the corners of a rectangular reference card. The registration marks may be for example circles. The registration marks are preferably of a known colour, for example blue circles are found to be particularly effective as they can be identified reliably in a range of lighting conditions. The registration marks are designed to be easy to identify compared with other features of the reference card.

Preferably, a de-skewing process is carried out by the processor based on the detected position of the registration marks. Typically, the registration marks on the reference card are at four corners of a rectangle. When a photograph is taken, the reference card may not be completely straight in the frame but the detected registration marks, as well as known characteristics of the original reference card, may be used to process the photograph to de-skew the image.

Once the image has been de-skewed, various features within the image may be determined by the processor primarily by reference to known information about the relative positions of features on the reference card and correctly-positioned dip test. Also, known edge detection algorithms may be used which together with the known relative positions can reliably identify the boundaries of colour change portion(s) of dip tester(s).

Identifying the area of the digital photograph corresponding to the colour-change portion of the dip test, and determining the colour of that area may comprise identifying a plurality of pixels corresponding to the colour-change portion of the dip tester, and determining the dominant colour among those pixels. The dominant colour is the most common single colour among the pixels.

Typically, to identify a plurality of pixels corresponding to a colour-change portion of the dip tester, the processor will by use of known information and edge detection find the boundary of the colour-change portion in the photograph, and then choose a sub-area completely within that boundary. It is the pixels within this sub-area that are then used to determine the dominant colour of the colour-change portion.

As an example, the sub-area may be for example 16×16 pixels square, containing 256 pixels in total. The dominant colour is the single most common colour among those 256 pixels, i.e. the mode average colour.

Preferably, the reference colours are provided as a substantially continuous colour gradient along one dimension of an area of the reference card. For example, a substantially rectangular colour gradient may be provided, substantially corresponding to the range of colours which the relevant dip-test can show, throughout the possible range of values of the characteristic being tested. In the example of a substantially rectangular colour gradient, the colour varies continuously in the dimension along the long side of the rectangle. Along an orthogonal direction, i.e. parallel to the short side of the rectangle, the colour is constant. To put it another way, the colour gradient rectangle is made up of a large number of adjacent lines, each line being a different colour.

The reference area(s) in the photograph may be identified, again, starting with known information as to the relative positions of areas of the reference card and preferably using edge detection algorithms. When the area of the photograph corresponding to a continuous gradient has been identified, it may be divided into sub-areas, each sub-area in principle being made up of a group of pixels corresponding to the same reference colour. Each sub-area may be an area a single pixel wide, and extending along multiple pixels along the direction where the reference colour is constant—e.g. parallel to the short side of the rectangle in the example where the colour changes along the long side of the rectangle. It will be appreciated that where the image is skewed—and any deskewing process is likely to be less than perfect—in fact these groups of pixels may extend somewhat along the colour gradient and therefore have pixels of different, but very similar, colours.

Within each sub-area of the reference area, a dominant colour may be identified, again by taking the single most common colour among all of the pixels.

Having determined a dominant colour of an area of the photograph corresponding to the colour-change portion of the dip test, and having identified dominant colours of the sub-areas of the reference area, a colour difference metric is calculated between the colour-change portion of the dip test and each of the sub-areas. The colour difference metric may be calculated according to the known LAB colour space difference system.

The reference colour with the least difference from the colour-change pad corresponds to the value of the characteristic being determined. The position of that reference colour in the image can be used to determine the correct value.

Preferably, a determination may be made as to whether the result thus obtained is valid, based on the series of difference values. In particular, the smoothness of the difference series along the reference gradient and the number of local minima may be used as threshold conditions to determine the validity of the result. If there are too many local minima in the difference series, or the series contains serious discontinuities or sharp changes, the result may be determined to be invalid. This could be because the quality of the photograph is poor due to bad lighting for example, and the test may be easily repeated in this case.

A Kalman filter may be applied to the difference series to smooth out noise, before the global minimum/least difference is determined, and/or before a determination as to validity of the result is made. A Kalman filter assists in more accurately reflecting the true colour differences of points along a gradient, by taking into account not only the measured differences between each point on the gradient and the colour-change portion, but also the expected relationship between differences associated with different points on the gradient due to the known characteristics of the gradient.

Once the value of the characteristic has been determined, it may be compared with predetermined threshold value(s) to produce a pass or fail test result. For example, for a “pass” the iron concentration must be less than a threshold value. Likewise for copper and aluminium concentration, typically a “pass” result will be for any concentration less than a threshold value. For pH, there will typically be lower and upper thresholds. For example, a “pass” result may be appropriate for a pH in the range 7.5-8.5.

Preferably, the colour gradient on the reference card includes portions of increased resolution in areas around the relevant threshold. For example, if the relevant pass threshold for iron concentration is less than 5 ppm (parts per million), then the colour gradient as printed on the card may include, for example, a 1 cm long section covering the range of colours indicating between 0 ppm and 4 ppm, a 3 cm long section covering the range of colours indicating between 4 ppm and 6 ppm, and a 1 cm long section covering the range of colours indicating between 6 ppm and 10 ppm. The precision of the test is therefore increased in the most relevant range, within which the “pass” or “fail” decision is made.

Where the colour reference card contains multiple colour references for multiple different dip tests, in some embodiments further validity checks may be made by comparing the colour of one colour-change pad with the colour reference gradients for other colour-change pads. For example, the colour-change pad for a test for copper concentration may be compared, as a validity check, to the reference gradient designed for the iron concentration dip test. If a closer match to the colour of the copper test pad is found on the iron reference gradient, then this may indicate an out-of-range value and the result of the test will be a fail, or invalid.

Preferably, multiple photographs are taken of each test, with the test result values being calculated for each photograph according to the procedure described above. The photographs may be taken under slightly different lighting conditions and from slightly different positions. This may have some impact on the test result values determined, which may therefore vary slightly for each photograph, even though all the photographs are of the same dip test and the same reference card. This gives an idea of the uncertainty in the result, and too much variance in the values thus obtained, especially if it crosses a pass/fail threshold, may cause an indication that the test is invalid. It has been found though that generally the variance is acceptably small, where lighting conditions are reasonable and the camera is of average quality such as is commonly provided with a modern mobile telephone. Where multiple values are obtained in this way, in some embodiments the final result value may be determined by taking a mean of the multiple values, and then choosing the single measured value closest to the mean.

Preferably, the whole process of taking a photograph or multiple photographs, and processing the photograph(s) to determine test result values and a pass or fail, is carried out on a mobile device, for example a mobile telephone or tablet computer. The device will typically include a camera, a processor and associated other parts of a computer, a display screen, and some form of user input. The mobile device runs a software program adapted to control the computer and various other devices to carry out the process of the invention.

In one embodiment, the mobile device running the software program may be adapted to continuously stream a feed from the camera to the display screen so that the user can “see what the camera sees”. Overlaid on the video stream on the display screen a template may be provided. The template is preferably the same shape as the colour reference card (for example, rectangular, with a particular aspect ratio). This helps the user to position the camera so that the colour reference card is more or less facing the camera directly, with minimal skew.

While the camera feed is being streamed to the display screen, the processor may continuously take pictures and process each one. For example, a typical video stream might be 30 or more frames per second. 30 pictures each second are therefore available for processing. Typically, the software will be adapted to silently drop frames when it is too busy to process them, so in reality fewer frames will be processed. However, multiple frames are continuously being processed, without any particular user intervention, while the user is holding the camera and positioning it as best he can so that the reference card and dip test is in the right position in the frame.

Each processed frame may be initially screened for sharpness. For example the Laplacian method may be used and images which are too blurry may be rejected.

With frames which pass the initial test for sharpness, the software is adapted to go on to look for the registration marks on the reference card. Typically, the registration marks are circles and are substantially the only circular features on the card. Circles can be detected by a process which may include a Gaussian blur to remove noise, an edge detection algorithm such as the Canny algorithm to detect edges, and then a removal of straight lines. This leaves candidate areas which may be circular registration marks. Typically, this may include some false positive areas which are not circles, but where distortion has caused what are really straight lines to not have been removed. A test to see if the area of the candidate circle is substantially greater than πr² is found to be effective to remove these “false circles”.

If the registration marks are found, the skew of the image can be calculated. Typically, the registration marks actually printed on the reference card will be at the corners of the rectangle. Normally, the registration marks detected in the photograph will not define a perfect rectangle but an irregular quadrilateral. Some degree of skewing is acceptable and can be corrected. However too much skewing will result in the photograph being rejected.

Typically, the screening for sharpness and skew goes on in the background while the image from the camera is continuously being screened to the display screen. No user intervention is required to capture particular frames. When a number of images which are acceptable in terms of sharpness and skew have been obtained (in a typical embodiment, 3-5 images) the software will stop streaming the camera feed to the display to indicate to the user that enough data has been captured and they no longer need to position the camera with respect to the reference card. Typically, in reasonable conditions with an average smartphone camera, the whole process may take a few seconds. The invention provides a reliable result which is not subject to the subjectivity of a human comparison. The result may simply be a “pass” or “fail”, based on threshold conditions. However, in some embodiments a “fail” result may include a recommendation of what treatment needs to be carried out on the central heating/cooling system.

The table below shows example conditions and associated recommendations which may be made based on test results.

| Component | Conditions | Result | Recommendation |
|---|---|---|---|
| Copper | Cu < 3.5 ppm | Pass | — |
| | 3.5 < Cu < 6.5 ppm | Pass | Above target level but |
| | | | compensated for by |
| | | | ADEY MC1 + Smart Chemistry. |
| | | | Maintain correct dosage |
| | | | levels. |
| | Cu > 6.5 ppm | Recommendation | Exceeds maximum target. |
| | | | Copper level is high, this can |
| | | | cause pitting corrosion of |
| | | | aluminium and |
| | | | steel. MagnaCleanse ® system |
| | | | flush is recommended. |
| Inhibitor | Mo ≥ 20.0 | Pass | Adey Protector detected |
| (Molybdenum) | Mo < 20, | Recommendation | Insufficient Adey Protector |
| | | | detected. Addition of |
| | | | Adey MC1 + is |
| | | | recommended. |
| Iron | Fe < 81 ppm | Pass | — |
| | If Mo > 50 ppm | Pass | — |
| | 81 < Fe < 125 ppm | | |
| | If Mo < 50 ppm | Recommendation | Additional ADEY Protector |
| | 81 < Fe < 125 ppm | | required to compensate for |
| | | | iron level. Install or clean |
| | | | ADEY MagnaClean ® filter. |
| | Fe > 125 ppm | Recommendation | Iron level is high indicating |
| | | | the presence of corrosion. |
| | | | System clean is |
| | | | recommended. Install or |
| | | | clean |
| | | | ADEY MagnaClean ® filter. |
| pH | 6.5 < pH < 8.5 | Pass | — |
| | pH < 6.5 | Recommendation | pH is low. Buffer by addition |
| | | | of ADEY Protector or system |
| | | | flush recommended. |
| | pH > 8.5 | Recommendation | pH is high. Buffer by addition |
| | | | of ADEY Protector or system |
| | | | flush recommended. |

Results may be stored in a central database, and/or email and/or paper reports, or reports in any other format, may be produced.

In some embodiments, the method may include capturing a photograph of a sample of the central heating/cooling system water being tested. The sample is preferably photographed against a light background. The photograph of the sample may be displayed on the screen of the device alongside several reference colours or ranges, for example three colours may be shown and the user may be asked which colour is most similar. This is a basic, and essentially manual, assessment of the turbidity of the water.

Although the invention is primarily envisaged for use in testing central heating or cooling system water, the method may also be adapted for dip testing of samples in other contexts. For example, swimming pool water is commonly dip tested to measure pH, chlorine levels, etc. Various industrial and other machines use water or other fluids which can be dip tested, and there are a multitude of potential applications in production machines, vehicles, shipping, residential, and commercial contexts.

### DESCRIPTION OF THE DRAWINGS

For a better understanding of the invention, and to show more clearly how it may be carried into effect, a preferred embodiment will now be described with reference to the accompanying drawings, in which:

FIG. 1 shows a dip tester stick including six colour change pads, together with a colour reference card, used as part of the invention;

FIG. 2 shows the dip tester stick and colour reference card of FIG. 1, with the dip tester stick positioned on the colour reference card according to reference indicia printed on the reference card;

FIG. 3 and FIG. 4 show the use of a mobile device to take pictures of the dip tester stick and colour reference card of FIG. 2;

FIG. 5 shows how a difference series may be obtained from areas of an image taken in FIG. 4; and

FIG. 6 shows an example of a difference series plotted on a graph.

### DESCRIPTION OF AN EMBODIMENT

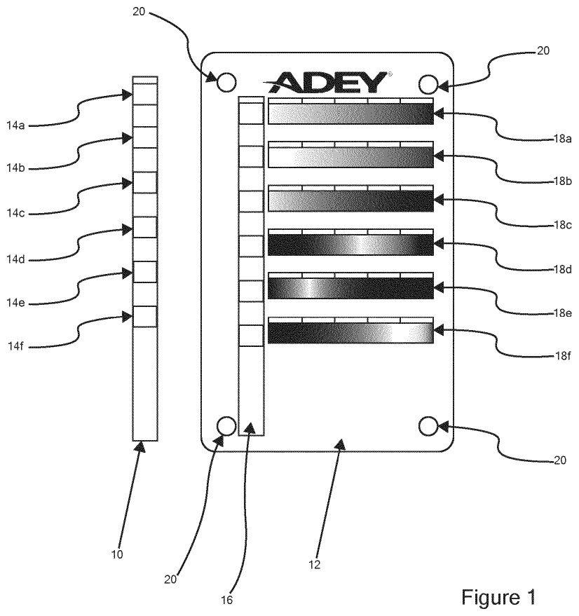

Referring firstly to FIG. 1, a dip tester stick is indicated at 10, next to a colour reference card indicated at 12. The dip tester stick includes six colour change pads 14a, 14b, 14c, 14d, 14e, 14f. Colour change pad 14a is impregnated with a colour change reagent which indicates the presence of a molybdate inhibitor. Colour change pad 14b is impregnated with a colour change reagent which indicates the presence of copper. Colour change pad 14c is impregnated with a colour change reagent which indicates the presence of iron. Colour change pads 14d, 14e, 14f are impregnated with colour change reagents which indicate the pH of the sample.

In other embodiments dip tester sticks may include five pads (for example, for testing molybdate, copper, iron, and two pads for pH), or four pads (for example, for testing molybdate, copper, iron, and one pad for pH).

The dip tester stick has been dipped for a few seconds in a sample of central heating and/or cooling water, and therefore the colour change pads have changed colour according to the characteristics of the central heating and/or cooling water which was sampled.

The colour reference card is printed with indicia 16 which show where the dip tester stick 10 is to be placed on the card, adjacent to colour reference gradients 18a, 18b, 18c, 18d, 18e, 18f.

In various embodiments, the dip tester may be placed on the card, or adjacent to the card, as long as the photograph is taken with the reference card and the dip tester in the same frame.

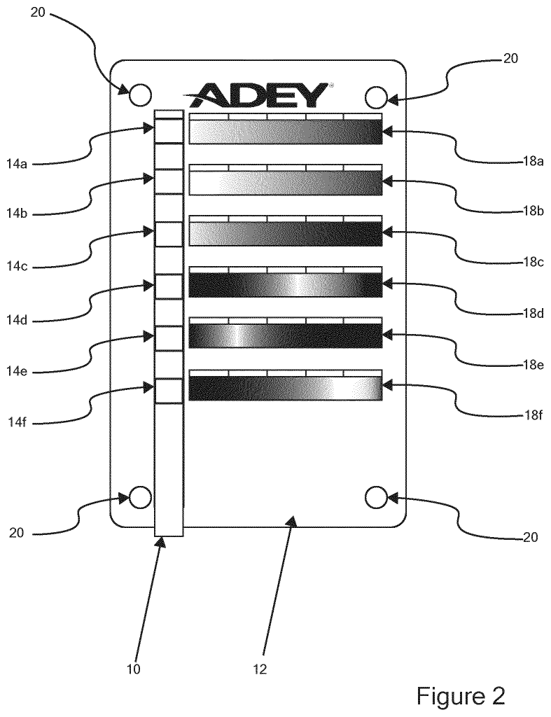

FIG. 2 shows the dip tester stick 10 placed on the reference card 12 in the location indicated by the indicia (16, FIG. 1). In this position, colour change pad 14a is adjacent colour reference gradient 18a, colour change pad 14b is adjacent colour reference gradient 18b, and so on.

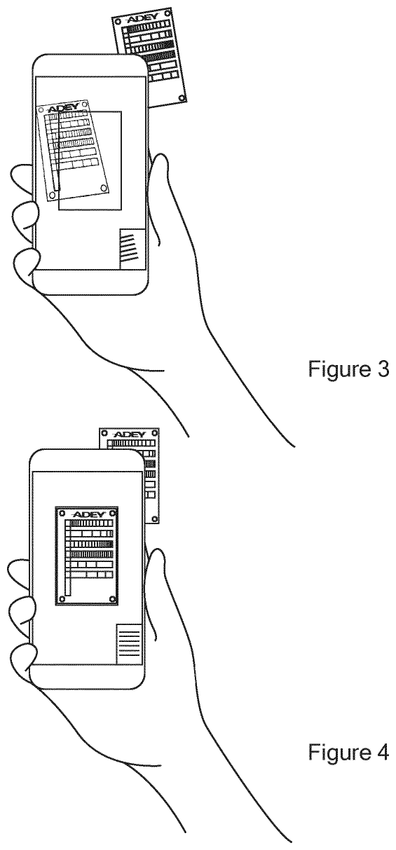

Referring now to FIG. 3, a mobile device is indicated at 100. The mobile device in this embodiment is a mobile telephone, but could be another suitable device such as a tablet computer. A suitable device has at least a camera, a display screen, and a processor. The mobile device 100 runs software which causes a template pattern 110 to be displayed on the display screen. The template pattern is the same shape as the reference card 12, i.e., in this embodiment, a rectangle with a certain ratio of the length of the long side to the length of the short side. The template pattern is displayed on the display screen overlaid on a direct video feed from the camera of the mobile device. The purpose of the template pattern is to assist the user in lining up the reference card in the camera's view, as closely as possible taking a photograph with minimal skew. In FIG. 3 there is some skew in the image on the mobile device, but the user can easily move the mobile device 100 to correct for this. FIG. 4 shows the mobile device 100 in more or less exactly an optimal position, with the image of the reference card 12 lined up exactly in the template pattern 110.

While the image from the camera is being continually streamed to the display, and the user is trying to adjust the position of the camera as best he can to line up the image of the reference card 12 with the template pattern 110, still photographs are continuously being taken and processed. Typically, the video stream from a mobile phone camera may be about 30 frames per second or more. As many individual frames as possible may be processed, with frames being silently dropped when the processor is too busy. Processing a frame may include an initial filtering stage to determine the sharpness of the image. Frames which are too blurry may be rejected. The Laplacian algorithm may be used as a known test for sharpness.

If a frame passes the sharpness test, the next stage is to check for the presence of expected registration marks. In this example, four registration marks 20 are provided, substantially at corners of the rectangular reference card 12. The registration marks are in the form of blue circles. The registration marks 20 are the only circular features on the reference card 12. In each image a process of identifying and filtering circles takes place. This typically comprises using an edge detection algorithm to identify and isolate features. For example, the Canny algorithm may be used. A Gaussian blur may first be applied to the image to reduce noise. Candidate circles can be identified firstly by removing straight lines. After straight lines are removed, remaining closed paths may be candidate circles. As a second stage/check, the area of each candidate feature can be measured (number of pixels inside the feature) and compared to a calculated area from a measured average radius of the candidate feature (by A=πr²). A candidate circle with a measured area of more than the calculated area is likely to be in reality, a square or another shape (bearing in mind that the edges may be pixelated and rough, this may not be obvious to the initial algorithm which finds candidate circle features). Therefore a candidate circle with a measured area more than the calculated area will be rejected and dropped from the set of candidate circles.

If a frame contains four detected registration marks, then the relative position of those registration marks is checked against predetermined constraints. On the original reference card 12, the registration marks 20 are at corners of a rectangle. The user interface as described above is designed to help the user to minimise skew, but in practice some small amount of skew is likely to be present in most processed images. The registration marks in the processed image will therefore usually not quite form a rectangle, but a trapezium (US: trapezoid). As long as the interior angles of the trapezium are close enough to right angles, within some predetermined tolerance, for example between 85 and 95 degrees, the image may be determined to be good enough for further processing. Whatever small skew is present in an acceptable image can be corrected in software using known techniques, based on the detected registration marks 20.

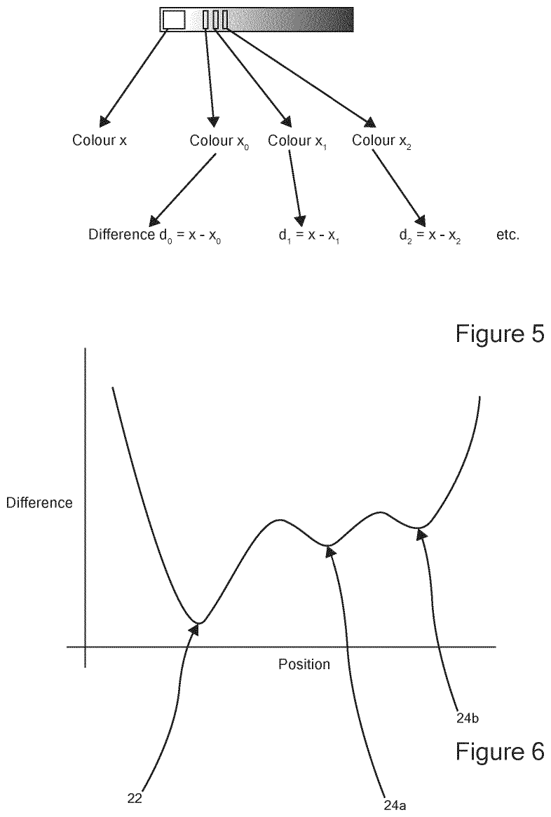

Referring now to FIG. 5, once an image has been selected as suitable and deskewed, different areas of the image can be identified with reference to known relative positions of the different components on the reference card (12). FIG. 5 shows an area of such an image, and black outlines show particular areas which are subject to individual processing. Firstly the (roughly square in this embodiment) area of the image corresponding to one of the colour change pads is identified. The relevant feature can be identified using an edge detection algorithm, and finding a feature in the right place on the image, using the identified registration marks (20) as reference points. A sub-area, entirely within the boundary of this identified feature, may be used for further processing. Excluding the edges of the identified feature leads to a patch with a more consistent colour throughout by removing boundary effects. Within the identified sub-area, which is marked in FIG. 5 by the black square outline, a dominant colour is identified. The dominant colour is the single most common colour of a pixel within the outline. In other words, it is the mode average pixel colour within that area of the image.

Dominant colours in strips of the colour reference gradients 18 are also identified in the same way. The position of the relevant colour reference strip 18 on the image is identified, and multiple sub-areas within the colour reference strip 18 are processed to find the dominant colour of each (the dominant colour again being the most common colour of pixel within the area). In this embodiment, the colour gradient changes continuously along the dimension running horizontally across FIG. 5. Along the other dimension, i.e. along the height of the reference strip, the colour is continuous. Therefore, by identifying sub-areas in the form of thin slices of the image of the colour reference strip 18, in principle every pixel in a given slice should be very similar or the same, with variations being due to artefacts from the process of taking a picture, the lighting involved, noise from the camera sensor, etc. These slices of the image of the reference strip are shown, to aid understanding, in FIG. 5. They are the three rectangular slices shown in black outline. It should be understood that the width of these slices is over-exaggerated in FIG. 5—typically in embodiments the slices may be only a single pixel wide. Also, in a real embodiment, the slices will usually be either abutting or very close to each other, with many hundreds of slices along the image of the reference gradient.

Once the dominant colour of the image of the coloured pad is identified, as well as the dominant colour of each of the strips of the image of the reference gradient, for each strip of the reference gradient a difference may be calculated between the dominant colour of that strip and the dominant colour of the colour-change pad. This leads to a difference series d₀ . . . dᵢ. FIG. 6 shows an example of a resulting series graphically, with the value of d on the vertical axis and the position of the relevant strip along the reference gradient on the horizontal axis. It is clear in FIG. 6 that there is a global minimum distance at 22. This is the position on the reference gradient which is closest to the colour of the colour change pad. From the position on the reference gradient which most closely matches the colour of the dip test, a characteristic of the heating and/or cooling water being sampled can be derived, for example, the concentration of iron in the sample.

The difference metrics may be calculated by the LAB colour space difference system.

In FIG. 6, there is a fairly clear global minimum 22. However, there are also local minima 24a, 24b in the difference series. A check may be made for validity of the result based on:
the number of local minima;
the difference between the global minimum and the second least local minimum (the second least local minimum is 24a in the FIG. 6 example);
a measure of the smoothness of the difference series;
the difference value of the global minimum found, which should not be too high.

If there are too many local minima, there is no sufficiently clear global minimum (i.e. the second least local minimum is only a slightly greater difference value than the global minimum), or if the curve has a low smoothness measure, the result may be determined to be invalid. This may lead to a repeat of the process of capturing photographs, or an indication to the user that the dip test itself needs to be repeated with a new dip tester, or an indication that a sample needs to be sent away for a lab test.

A Kalman filter may be applied to the difference series before the global minimum is determined and the various checks for validity are made.

Typically, the initial process of capturing and screening frames may be repeated until several acceptable frames are obtained. A measure of the characteristic (e.g. concentration of iron) is made on each captured frame. The final determination is preferably made by taking a mean of the values obtained from each frame, and then reporting the single value which is closest to the mean. If there is too much variance in the values obtained from the different frames, then the result may be determined to be invalid.

FIG. 5 shows a single colour change pad and reference pattern, taken from an image of the card shown in FIG. 2 with six colour change pads 14a-f and six reference gradients 18a-f. The process of determining the difference series and thus the measured value of each characteristic is repeated for the other five pairs of reference gradients and colour-change pads. However, in some embodiments, comparisons may also be made between the colour of a colour-change pad, and reference gradients other than the reference gradient corresponding to the colour-change pad. As a further check, if a colour-change pad is closer in colour to any point in a non-corresponding reference gradient, then the result may be determined to be invalid. It is likely in this case that the characteristic being measured by the colour change pad is completely out of the range envisaged by the reference gradient. For example, an extremely high concentration of iron might lead to a dark brown colour-change pad, closer in colour to one of the reference gradients corresponding to the pH colour-change pads.

The invention described can be used to very easily and accurately test central heating and/or cooling water for a range of characteristics. By using an ordinary mobile telephone running appropriate software, an accurate determination of values of various characteristics can be made. The accuracy achieved is comparable with, or better than, experienced human assessment of chemical dip tests. Moreover, various checks are in place to determine when the result obtained can be relied on and when it cannot. In the worst case therefore, the method of the invention will reach the conclusion that it cannot determine the relevant characteristics of the sample, in contrast with some prior art colour matching systems which often return an incorrect result.

1. A method of testing central heating and/or cooling water comprising the steps of:
providing at least one color-change dip tester for measuring a characteristic to be tested, the color-change dip tester having a color-change pad, and dipping the color-change dip tester in the central heating and/or cooling water;
providing a color reference card, the color reference card having a range of reference colors on its surface, the reference colors on the color reference card corresponding to a range of possible colors of a dipped color tester;
using a digital camera, taking a photograph of the dipped dip tester and the color reference card;
by a processor, identifying an area of the digital photograph corresponding to an image of the color-change pad of the dip tester, and determining a color of that area of the photograph;
by the processor, identifying areas in the digital photograph corresponding to an image of the reference colors on the color reference card, and determining colors associated with reference areas;
determining the closest of the colors associated with reference areas to the color of the image of the color-change pad of the dip tester, and determining on that basis a value of the characteristic to be tested.

2. The method of testing central heating and/or cooling of claim 1, wherein the characteristic to be tested is at least one of pH, concentration of iron, concentration of copper, concentration of aluminum, and concentration of a corrosion inhibitor.

3. The method of testing central heating and/or cooling water of claim 1, wherein the color reference card includes indicia indicating a position in which the dip tester may be placed on or adjacent to the reference card, and in which the dip tester is located in the indicated position before the photograph is taken.

4. The method of testing central heating and/or cooling water of claim 1, wherein the color reference card includes registration marks.

5. The method of testing central heating and/or cooling water of claim 1, wherein identifying the area of the digital photograph corresponding to the color-change pad of the dip test and determining the color of that area comprises identifying a plurality of pixels corresponding to the color-change pad of the dip tester and determining the single most common color among those pixels.

6. The method of testing central heating and/or cooling water of claim 1, wherein identifying areas of the digital photograph corresponding to reference areas and determining the color of the reference areas comprises identifying a plurality of pixels corresponding to each reference area, and determining the single most common color among each plurality of pixels.

7. The method of testing central heating and/or cooling water of claim 1, further comprising determining a difference series based on the differences between the color of the area of the image corresponding with the color-change pad and the color of each of the reference areas.

8. The method of testing central heating and/or cooling water—of claim 7, wherein a Kalman filter is applied to the difference series.

9. The method of testing central heating and/or cooling water of claim 7, wherein a determination as to validity of the determined value of the characteristic is made based on a measure of the smoothness of the difference series.

10. (canceled)

11. The method of testing central heating and/or cooling of claim 1, wherein a pass or fail result is output based on a comparison of the determined value of the characteristic with a predetermined threshold value.

12. The method of testing central heating and/or cooling water of claim 11, wherein the color gradient on the reference card includes portions of increased resolution in areas around the color corresponding to the predetermined threshold.

13. The method of testing central heating and/or cooling of claim 1, wherein color-change pads are provided, and multiple corresponding reference gradients are provided on the reference card.

14. The method of testing central heating and/or cooling water of claim 13, wherein a determination as to the validity of the determined value of a characteristic is made based on comparing the color of the color-change pad corresponding to that characteristic with a color reference gradient corresponding to a different characteristic.

15. The method of testing central heating and/or cooling water of claim 1, wherein a mobile device including a camera, a display screen, and a processor is provided, the mobile device being adapted to stream images from the camera to the display screen, and superimpose a template pattern on the display screen to aid in positioning the camera relative to the reference card and dip tester.

16. The method of testing central heating and/or cooling water of claim 15, wherein the mobile device is adapted to continuously process frames from the camera stream, at the same time as the camera stream is being displayed on the display screen, and to filter frames for suitability.

17. The method of testing central heating and/or cooling water of claim 16, wherein the filter for suitability includes a test against a sharpness threshold.

18. The method of testing central heating and/or cooling water of claim 16, wherein the color reference card includes registration marks and in which the filter for suitability includes a test as to whether registration marks can be detected in expected locations.

19. The method of testing central heating and/or cooling of claim 16, wherein the mobile device is adapted to stop streaming the camera feed to the display when more than a predetermined number of acceptable frames have been captured.

20. The method of testing central heating and/or cooling water of claim 15, wherein the mobile device is a mobile telephone or a tablet computer.

21. (canceled)

22. A non-transitory computer readable medium containing instructions which when executed on a processor of a mobile device, the mobile device including a camera, a display screen and a processor, cause the mobile device to carry out the steps of the method of claim 1 on a dipped color-change dip tester and associated color reference card.

23-25. (canceled)
